(19) United States
(12) Patent Application Publication
(10) Pub. No.: US 2011/0269964 A1
(43) Pub. Date: Nov. 3, 2011

(54) N-Alkylation of Opiates

(75) Inventors: Jian Bao, Chesterfield, MO (US); Catherine E. Thomasson, Webster Groves, MO (US); Esa T. Jarvi, Ballwin, MO (US)

(73) Assignee: Mallinckrodt Inc., Hazelwood, MO (US)

(21) Appl. No.: 13/095,952

(22) Filed: Apr. 28, 2011

Related U.S. Application Data
(60) Provisional application No. 61/329,118, filed on Apr. 29, 2010.

(51) Int. Cl. C07D 489/00

(52) U.S. Cl. 546/45; 546/46

(57) ABSTRACT

The present invention provides an efficient process for preparing N-alkylated opiates. In particular, processes are provided for using a chloride-containing alkylating agent and a bromide or iodide salt to alkylate the corresponding nor-opiate.

### CROSS REFERENCE TO RELATED APPLICATIONS

This application claims the benefit of U.S. Provisional Application No, 61/329,118 filed Apr. 29, 2010, which is incorporated herein in its entirety.

### FIELD OF THE INVENTION

The present invention generally relates to the preparation of N-alkylated opiates. Specifically, the invention relates to the use of a chloride-containing alkylating agent and a bromide or iodide salt to alkylate the corresponding nor-opiate.

### BACKGROUND OF THE INVENTION

Naltrexone is an opioid receptor antagonist that is primarily used in the management of alcohol dependence and opioid dependence. Historic procedures for the synthesis of naltrexone and other N-alkylated opiates involve using an alkyl bromide to alkylate the corresponding nor-opiate (e.g., noroxymorphone). Among the disadvantages of these procedures include low yields (because of significant side reactions), difficulty in handling the reactants, instability of alkyl bromides, as well as the high cost of alkyl bromides. Synthesis procedures have also been developed for synthesizing naltrexone from other starting materials (e.g., oripavine) because of the insolubility of noroxymorphone, but these processes are multi-stepped and, thus, more expensive. Therefore, there is a need for an improved, high yielding, one-step process for the preparation of naltrexone and other N-alkylated opiates from the corresponding nor-opiate in which a cheaper and/or more stable alkylating agent is employed.

### SUMMARY OF THE INVENTION

Among the various aspects of the present invention is the provision of a process for preparing a compound comprising Formula (II):

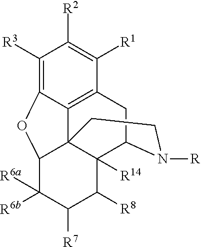

The process comprises contacting a compound comprising Formula (I) with R-chloride and a bromide or iodide salt to form the compound comprising Formula (II). The compound comprising Formula (I) has the following structure:

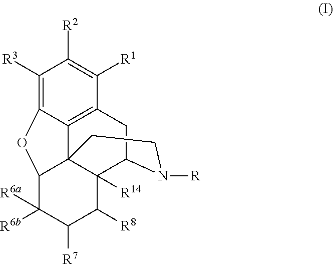

wherein:
R is chosen from hydrocarbyl and substituted hydrocarbyl;
R¹ and R² are independently chosen from hydrogen, halogen, {—}OR¹¹, hydrocarbyl, and substituted hydrocarbyl;
R³, R⁷, and R⁸ are independently chosen from hydrogen, {—}OR¹¹, hydrocarbyl, and substituted hydrocarbyl;
R^{6a} and R^{6b} are independently chosen from hydrogen, {—}OR¹¹, hydrocarbyl, substituted hydrocarbyl, combining with R¹⁴ to from an alkeno bridge, and together forming {═}O or {═}CH₂;
R¹¹ is chosen from hydrogen, hydrocarbyl, substituted hydrocarbyl, and a hydroxy protecting group; and
R¹⁴ is chosen from hydrogen, {—}OR¹¹; hydrocarbyl, substituted hydrocarbyl, and combining with R^{6a} or R^{6b} to form an alkeno bridge.

Other aspects and features of the invention are described in more detail below.

### DETAILED DESCRIPTION

The present invention provides an improved synthetic process for the preparation of an N-alkylated opiate from the corresponding nor-opiate. The process comprises reacting the nor-opiate with a chloride-containing alkylating agent (i.e., R-chloride) and a bromide or iodide salt to form the N-alkylated opiate. For example, noroxymorphone may be reacted with cyclopropylmethyl chloride or allyl chloride in the presence of a bromide salt to form naltrexone or naloxone, respectively. An advantage of the process is the use of chloride-containing alkylating agents, which are quite stable, reasonably priced and readily available. Because of their stability, however, they are not very reactive. The chloride-containing alkylating agent, however, may be activated via a halogen exchange reaction by contact with a molar excess of a bromide or iodide salt. Contrary to conventional wisdom, catalytic amounts of bromide salt failed to activate R-chloride because little or no product was made (see Examples 1 and 2). In contrast, when a molar excess of bromide salt was used, R-chloride was activated and the reaction went to completion (see Example 3). A further advantage of the process is that the yield of the product is greatly improved relative to current processes because side reactions are inhibited and the reactants are easier to handle (e.g., there is less gumming during the reaction).

### (I) Process for the Preparation of a Compound Comprising Formula (II)

Provided herein is a process for preparing an N-alkylated opiate, i.e., a compound comprising Formula (II). Specifically, the process comprises contacting a compound comprising Formula (I) with R-chloride and a bromide or iodide salt to form the compound comprising Formula (II). For the purposes of illustration, the following reaction scheme depicts this aspect of the invention:

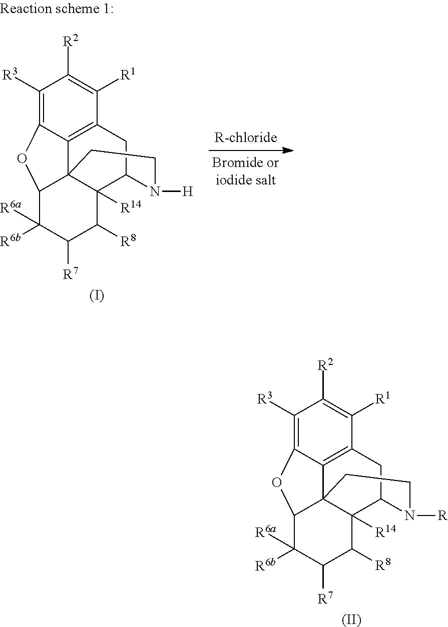

wherein:
R is chosen from hydrocarbyl and substituted hydrocarbyl;
R¹ and R² are independently chosen from hydrogen, halogen, {—}OR¹¹, hydrocarbyl, and substituted hydrocarbyl;
R³, R⁷, and R⁸ are independently chosen from hydrogen, {—}OR¹¹, hydrocarbyl, and substituted hydrocarbyl;
R^{6a} and R^{6b} are independently chosen from hydrogen, {—}OR¹¹, hydrocarbyl, substituted hydrocarbyl, combining with R¹⁴ to from an alkeno bridge, and together forming {═}O or {═}CH₂;
R¹¹ is chosen from hydrogen, hydrocarbyl, substituted hydrocarbyl, and a hydroxy protecting group; and
R¹⁴ is chosen from hydrogen, {—}OR¹¹; hydrocarbyl, substituted hydrocarbyl, and combining with R^{6a} or R^{6b} to form an alkeno bridge.

In one embodiment, R may be alkyl, alkenyl, alkynyl, aryl, substituted alkyl, substituted alkenyl, substituted alkynyl, or substituted aryl. In various iterations, R may be methyl, ethyl, propyl, butyl, cycloalkyl, cyclopropylmethyl, cyclobutylmethyl, allyl, propargyl, or benzyl.

In another embodiment, R¹, R², and R⁸ may be hydrogen. In a further embodiment, R³ may be alkoxy, hydroxy, or protected hydroxy. In a further embodiment, R^{6a} and R^{6b} together may form {═}O or {═}CH₂, or one of R^{6a} or R^{6b} may by hydroxy or methoxy and the other may be hydrogen; or one of R^{6a} or R^{6b} may combine with R¹⁴ to form an ethano bridge and the other may be methoxy. In yet another embodiment, R⁷ may be hydrogen or a substituted alkyl, such as, e.g., {—}CH(CH₃)(OH)CH(CH₃)₃. In yet another embodiment, R¹⁴ may be hydrogen, hydroxy, or combine with R^{6a} or R^{6b} to form an ethano bridge.

In a further embodiment, R may be cyclopropylmethyl or allyl; R¹, R², R⁷ and R⁸ may be hydrogen; R³ may be hydroxyl; R^{6a} and R^{6b} together may form {═}O; and R¹⁴ may be hydroxy. In an alternate embodiment, R may be cyclopropylmethyl; R¹, R², and R⁸ may be hydrogen; R³ may be hydroxyl; one of R^{6a} or R^{6b} may combine with R¹⁴ to form an ethano bridge and the other may be methoxy, and R⁷ may be {—}CH(CH₃)(OH)CH(CH₃)₃.

### (a) Reaction Mixture

The process commences with formation of a reaction mixture comprising the compound comprising Formula (I), the chloride-containing alkylating agent (i.e., R-chloride), and the bromide or iodide salt. As detailed below, the reaction mix may further comprise a proton acceptor and a solvent.

(i) Chloride-Containing Alkylating Agent (R-Chloride)

A variety of chloride-containing alkylating agents are suitable for use in the process. Suitable R-chlorides include alkyl chlorides, alkenyl chlorides, alkynyl chlorides, aryl chlorides, hydroxyalkyl chlorides, aryl alkyl chlorides, carboxyalkyl chlorides, and (alkyloxycarbonyl)alkyl chlorides. In various embodiments, R-chloride may be, without limit, methyl chloride, ethyl chloride, cyclopropylmethyl chloride, cyclobutylmethyl chloride, vinyl chloride, allyl chloride, benzyl chloride, and propargyl chloride. In one embodiment, R-chloride may be cyclopropylmethyl chloride. In another embodiment, R-chloride may be allyl chloride.

The molar ratio of the compound comprising Formula (I) to R-chloride can and will vary. In general, the molar ratio of the compound comprising Formula (I) to R-chloride will range from about 1:0.5 to about 1:2.5. In various embodiments, the molar ratio of the compound comprising Formula (I) to R-chloride may be about 1:0.8, 1:0.9, 1:1, 1:1.1, 1:1.2, 1:1.3, 1:1.4, 1:1.5, 1:1.6, 1:1.7, 1:1.8, 1:1.9, or 1:2. In some embodiments, the molar ratio of the compound comprising Formula (I) to the alkyl chloride may be about 1:1.2, 1:1.3, or 1:1.4.

In some embodiments, the total amount of R-chloride to be added may be divided into smaller aliquots and added in portions. In other embodiments, the total mount of R-chloride to be added may be added all at once.

(ii) Bromide or Iodide Salt

The reaction mixture further comprises a bromide or iodide salt. Without being bound to any particular theory, the bromide or iodide salt may serve to activate the chloride-containing alkylating reagent via a halogen exchange reaction. A variety of bromide or iodide salts may be used in the process. The bromide or iodide salt may be inorganic or organic. Non-limiting examples of suitable inorganic salts include sodium bromide or iodide salts, potassium bromide or iodide salts, lithium bromide or iodide salts, magnesium bromide or iodide salts, and the like. Suitable examples of organic bromide or iodide salts include quaternary ammonium compounds comprising R¹R²R³R⁴N⁺, wherein R¹, R², R³, and R⁴ are independently alkyl. Non-limiting examples of such organic salts include tetramethylammonium bromide or iodide salts, tetraethylammonium bromide or iodide salts, tetrapropylammonium bromide or iodide salts, tetrabutylammonium bromide or iodide salts, dimethylethylammonium bromide or iodide salts, dimethylpropylammonium bromide or iodide salts, tributylmethylammonium bromide or iodide salts, and so forth. In one embodiment, the bromide or iodide salt may be sodium bromide. In another embodiment, the bromide or iodide salt may be tetramethylammonium bromide or tetrabutylammonium bromide. In a further embodiment, the bromide or iodide salt may be a mixture of sodium bromide and an alkylammonium bromide.

The amount of bromide or iodide salt utilized in the process can and will vary, provided that at least 1.1 equivalents are used. In general, the molar ratio of the compound comprising Formula (I) to bromide or iodide salt will range from about 1:1.1 to about 1:5. In certain embodiments, the molar ratio of the compound comprising Formula (I) to bromide or iodide salt may be about 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, or 1:3.2. In one embodiment, the molar ratio of the compound comprising Formula (I) to bromide or iodide salt may be about 1:2.9.

(iii) Proton Acceptor

The reaction typically is conducted in the presence of a proton acceptor. The proton acceptor generally has a pKa between about 7 and about 13. Suitable proton acceptors having this characteristic include borate salts (such as, for example, NaBO₃), di- and tri-basic phosphate salts (such as, for example, Na₂HPO₄ and Na₃PO₄, and the like), bicarbonate salts (such as, for example, NaHCO₃, KHCO₃, LiHCO₃, and the like), carbonate salts (such as, for example, Na₂CO₃, K₂CO₃, Li₂CO₃, and the like), organic bases (such as, for example, an amine comprising R¹R²R³N wherein R¹, R², and R³ are independently alkyl; pyridine; N,N dimethylaminopyridine, etc.), and mixtures of any of the above. In some embodiments, the proton acceptor may be sodium bicarbonate, sodium carbonate, sodium phosphate, potassium bicarbonate, potassium carbonate, potassium phosphate, triethylamine, diisopropylethyl amine, or combinations thereof. In one embodiment, the proton acceptor may be a mixture of sodium bicarbonate and sodium carbonate.

The amount of proton acceptor utilized in the process can and will vary. In general, the molar ratio of the compound comprising Formula (I) to the proton acceptor will range from about 1:1 to about 1:4. In some embodiments, the molar ratio of the compound comprising Formula (I) to the proton acceptor may be about 1:1, 1:1.5, 1:2, 1:2.5, 1:3, 1:3.5, or 1:4. In one embodiment, the molar ratio of the compound comprising Formula (I) to the proton acceptor may be about 1:2.

In general, the pH of the reaction mixture is adjusted such that the reaction is conducted at a pH from about 8 to about 10. In various embodiments, the pH of the reaction may be about 8.0, 8.25, 8.5, 8.75, 9.0, 9.25, 9.5, 9.75, or 10.0. In one embodiment, the pH of the reaction may range from about 9.0 to about 9.5. Those of skill in the art are familiar with appropriate pH adjusting agents. Non-limiting examples of suitable pH adjusting agents include ammonium hydroxide, potassium hydroxide, sodium hydroxide, and the like, as well as acetic acid, hydrochloric acid, sulfuric acid, and so forth.

(iv) Solvent

The reaction generally is conducted in the presence of a solvent. In general, the solvent will be an acetamide solvent. Non-limiting examples of suitable acetamide solvents include N,N-dimethylformamide (DMF), N,N-dimethylacetamide (DMAC), N-methyl-2-pyrrolidinone (NMP), and combinations thereof. In some other embodiments, the acetamide solvent may be part of a solvent mixture that further comprises another solvent. Non-limiting example of suitable solvents include acetonitrile, propionitrile, alcohols, water, and combinations thereof. In certain embodiments, the solvent mixture may comprise the acetamide solvent and water. The concentration of water in the solvent mixture can and will vary. In general, the amount of water in the solvent mixture may range from about 1% by weight to about 15% by weight. In various embodiments, the amount of water in the solvent mixture may be about 4%, 5%, 6%, 7%, 8%, 9%, 10%, 11%, or 12% by weight. In one embodiment, the amount of water in the solvent mixture may range from about 5% to about 10% by weight.

The amount of solvent utilized in the reaction can and will vary. In general, the weight ratio of the solvent or solvent mixture to the compound comprising Formula (I) will range from about 2:1 to about 10:1. In some embodiments, the weight ratio of the solvent or solvent mixture to the compound comprising Formula (I) may be about 3:1, 3.25:1, 3.5:1, 3.75:1, 4:1, 4.25:1, 4.5:1, 4.75:1 or 5:1. In one embodiment, the weight ratio of the solvent or solvent mixture to the compound comprising Formula (I) may range from about 3:1 to about 4:1.

### (b) Reaction Conditions

In general, the reaction will be conducted at a temperature ranging from about 20° C. to about 120° C. In certain embodiments, the temperature of the reaction may range from about 20° C. to about 40° C., from about 40° C. to about 70° C., or from about 70° C. to about 100° C. In one embodiment, the temperature of the reaction may range from about 80° C. to about 90° C. In another embodiment, the temperature of the reaction may be about 30° C. Typically, the reaction is conducted under ambient pressure and under an inert atmosphere (e.g., nitrogen or argon).

The duration of the reaction can and will vary. Typically, the reaction may proceed for a period of time ranging from about 1 hour to about 24 hours. In some embodiments, the duration of the reaction may range from about 2 hours to about 4 hours, from about 4 hours to about 12 hours, or from about 12 hours to about 24 hours. In one embodiment, the reaction may be allowed to proceed for about 3 hours. In another embodiment, the reaction may be allowed to proceed for about 16 hours. In general, the reaction is allowed to proceed for a sufficient period of time until the reaction is substantially complete, as determined by any method known to one skilled in the art, such as chromatography (e.g., HPLC). In this context, a “completed reaction” generally means that the reaction product mixture contains a significantly diminished amount of the compound comprising Formula (I) and a significantly increased amount of the compound comprising Formula (II) compared to each at the beginning of the reaction. Generally, the amount of the compound comprising Formulas (I) remaining in the reaction mixture may be less than about 3%, and preferably less than about 1%.

The yield of the compound comprising Formula (II) can and will vary. Typically, the yield of the compound comprising Formula (II) will be at least about 60%. In various embodiments, the yield of the compound comprising Formula (II) may range from about 60% to about 70%, from about 70% to about 80%, or from about 80% to about 90%. In still another embodiment, the yield of the compound comprising Formula (II) may be greater than about 90%, greater than about 95%, greater than about 98%, or greater than about 99.5%.

Upon completion of the reaction, the compound comprising Formula (II) may be isolated from the reaction mixture using techniques familiar to those of skill in the art. Non-limiting examples of suitable techniques include precipitation, extraction, filtration, distillation, crystallization, and the like. The final product may be washed and dried, and analyzed by HPLC, HPLC, MS, NMR, IR, or TGA.

The compound comprising Formula (II) may be converted to a pharmaceutically acceptable salt using standard techniques. Pharmaceutically acceptable salts include, without limitation, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

### (c) Stereochemistry

The compounds comprising Formulas (I) or (II) may have a (−) or (+) orientation with respect to the rotation of polarized light. More specifically, each chiral center of the compound may have an R or an S configuration. For the purposes of illustration, the ring atoms of the core morphinan structure are numbered as diagrammed below:

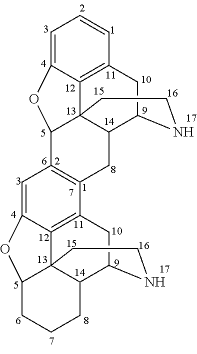

The compounds described herein have at least four chiral centers, namely carbons C-5, C-9, C-13, and C-14. At each chiral center, the stereochemistry at the carbon atom is independently R or S. The configuration of C-5, C-9, C-13, and C-14, respectively, may be RRRR, RRRS, RRSR, RSRR, SRRR, RRSS, RSSR, SSRR, SRRS, SRSR, RSRS, RSSS, SRSS, SSRS, SSSR, or SSSS, provided that the C-15 and C-16 atoms are both on the alpha face of the molecule or both on the beta face of the molecule.

In embodiments in which the compounds comprising Formulas (I) or (II) have a (−) orientation, the configuration of C-5, C-13, C-14, and C-9, respectively, may be RSRR. In embodiments in which the compounds comprising Formulas (I) or (II) have a (+) orientation, the configuration of C-5, C-13, C-14, and C-9, respectively, my be SRSS.

### (II) Process for the Preparation of a Compound Comprising Formula (IIa)

Another aspect of the invention encompasses a process for preparing a compound comprising Formula (IIa) from a compound comprising Formula (Ia), as depicted in the following reaction scheme:

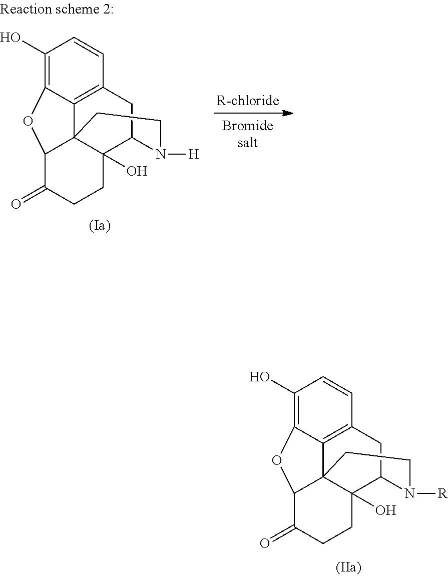

wherein:
R is cyclopropylmethyl or allyl.

The method comprises contacting the compound comprising Formula (Ia) with cyclopropylmethyl chloride or allyl chloride and a bromide salt to form the compound comprising Formula (IIa). The bromide salt may be sodium bromide or a mixture of sodium bromide and an alkylammonium bromide. Suitable concentrations of R-chloride and bromide salt are detailed above in sections (I)(a)(i) and (I)(a)(ii), respectively. In general, the reaction is conducted in the presence of a proton acceptor and a solvent as described in sections (I)(a)(iii) and (I)(a)(iv), respectively, and under reaction conditions as detailed in section (I)(b).

In one embodiment, molar ratio of the compound comprising Formula (Ia) to cyclopropylmethyl chloride or allyl chloride to bromide salt may range from about 1:1.4:2 to about 1:1.6:3. The proton acceptor may be sodium bicarbonate, sodium carbonate, triethylamine, diisopropyl ethyl amine, or combinations thereof. The molar ratio of the compound comprising Formula (I) to the proton acceptor may be about 1:2. The solvent may be N,N-dimethylformamide (DMF), N,N-dimethylacetamide (DMAC), or N-methyl-2-pyrrolidinone (NMP), wherein the solvent may further comprise about 5-15% of water by weight. The weight ratio of the solvent to the compound comprising Formula (Ia) may range from about 3:1 to about 4:1. The temperature of the reaction may range from about 30° C. to about 90° C., and the yield of the compound comprising Formula (IIa) may be at least about 80% by weight.

### DEFINITIONS

To facilitate understanding of the invention, several terms are defined below.

The term “acyl,” as used herein alone or as part of another group, denotes the moiety formed by removal of the hydroxy group from the group COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups having at least one double bond and that preferably contain from two to eight carbon atoms in the principal chain and up to 20 carbon atoms. They may be straight or branched chain or cyclic and include ethenyl, propenyl, isopropenyl, butenyl, isobutynyl, hexenyl, and the like.

The term “alkynyl” as used herein describes groups having at least one triple bond and that preferably contain from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “protecting group” as used herein denotes a group capable of protecting an oxygen atom (and hence, forming a protected hydroxy), wherein the protecting group may be removed, subsequent to the reaction for which protection is employed, without disturbing the remainder of the molecule. Exemplary protecting groups include ethers (e.g., allyl, triphenylmethyl (trityl or Tr), p-methoxybenzyl (PMB), p-methoxyphenyl (PMP)), acetals (e.g., methoxymethyl (MOM), β-methoxyethoxymethyl (MEM), tetrahydropyranyl (THP), ethoxy ethyl (EE), methylthiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Trot), 2-trimethylsilylethyl carbonate), silyl ethers (e.g., trimethylsilyl (TMS), triethylsilyl (TES), triisopropylsilyl (TIPS), triphenylsilyl (IPS), t-butyldimethylsilyl (TBDMS), butyldiphenylsilyl (TBDPS) and the like. A variety of protecting groups and the synthesis thereof may be found in “Protective Groups in Organic Synthesis” by T. W. Greene and P. G. M. Wuts, John Wiley & Sons, 1999.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Having described the invention in detail, it will be apparent that modifications and variations are possible without departing from the scope of the invention defined in the appended claims.

### EXAMPLES

The following examples are included to demonstrate various embodiments of the invention. It should be appreciated by those of skill in the art that the techniques disclosed in the examples represent techniques discovered by the inventors to function well in the practice of the invention. Those of skill in the art, however, in light of the present disclosure, should appreciate that many changes can be made in the specific embodiments that are disclosed and still obtain a like or similar result without departing from the spirit and scope of the invention, therefore all matter set forth is to be interpreted as illustrative and not in a limiting sense.

### Examples 1-18

### Preparation of Naltrexone

To prepare naltrexone, a bromide salt was used to activate chloromethylcyclopropane via an exchange reaction. The following reaction scheme depicts the synthesis of naltrexone:

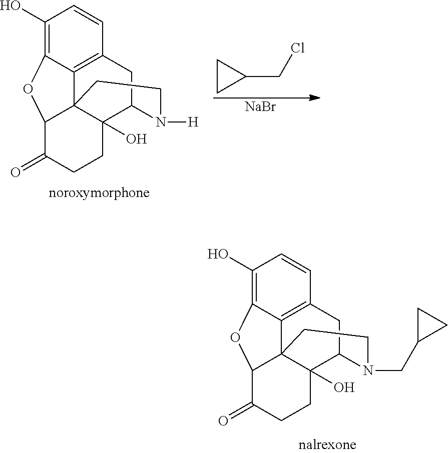

### Example 1

### Preparation of Naltrexone Using a Catalytic Amount of a Bromide Source—Trial 1

Noroxymorphone (0.50 g, 1.74 mmol) and NaBr (0.348 mmol, 0.2 eq) were added to a three neck flask followed by 4 mL of dimethylacetamide (DMAC). NaHCO₃ (0.31 g, 3.65 mmol, 2.1 eq) and chloromethylcyclopropane (0.23 g, 2.61 mmol, 1.5 eq) were also added. The mixture was stirred under N₂ and put in an oil bath heated to 120° C. In three hours, a sample was taken. Chromatography showed that the conversion was only partial. A majority of the noroxymorphone (>80%) was still present, and little naltrexone (<20%) was formed. Impurities, including 3-O-alkylated products, increased over the time.

### Example 2

### Preparation of Naltrexone Using a Catalytic Amount of a Bromide Source—Trial 2

Noroxymorphone (0.50 g, 1.74 mmol) and Bu₄NBr (0.112 g, 0.348 mmol, 0.2 eq) were added to a three neck flask followed by 4 mL of DMAC, NaHCO₃ (0.31 g, 3.65 mmol, 2.1 eq) and chloromethylcyclopropane (0.23 g, 2.61 mmol, 1.5 eq) were also added. The mixture was stirred under N₂ and put in an oil bath heated to 120° C. After five hours, a sample was taken. Chromatography showed that the conversion was incomplete. Naltrexone peak area was only about 60% and the related impurities especially the 3-O-alkylated products (>6%) was greater than 10%.

### Example 3

### Preparation of Naltrexone Using an Excess of a Bromide Source

Noroxymorphone (7.0 g, 24.4 mmol), NaBr (7.0 g, 2.8 eq), and a mixture of 48.8 mmol of base [i.e., Na₂CO₃ (1.81 g) and NaHCO₃ (2.66 g)], were added to a flask. 28 mL of solvent (119 (v/v), water/N-methyl-pyrrolidone, NMP) was added to the flask. Then, chloromethylcyclopropane (3.11 mL, 1.38 eq) was added in portions. The mixture was heated to 80° C. At 3 hours, a sample was taken. Chromatography revealed that only 1.5% of the starting noroxymorphone remained, while greater than 95% of naltrexone was formed. Related impurities were 2.2%.

### Example 4

### Parameter Optimization

A set of design optimization experiments was conducted in which the reaction time, percentage of water in the NMP solvent mixture, amount of sodium bromide, amount of chloromethylcyclopropane (CPMC), and amount of optional Na₂HPO₄ were varied (see Table 1). All reaction mixtures contained 3 g of noroxymorphone, 15 mL of a solvent mixture, and 1.5 eq. of Hunig's base (i.e., diisopropyl ethylamine). The reactions were conducted at 80-84° C. Table 1 presents the ratio of naltrexone formed and the amounts of impurities.

TABLE 1
Parameter Optimization

| | | | | | | | | | N- |
|---|---|---|---|---|---|---|---|---|---|
| | | | | | | | 3-O | Total | butenyl |
| Center | Time | Water | NaBr | Na₂HPO₄ | CPMC | Naltrexone/ | Impurity | Impurity | Impurity |
| Point | (hr) | (%) | (eq) | (eq) | (eq) | Noroxymorphone | (%) | (%) | (%) |
| 1 | 4 | 15 | 2 | 0 | 1.6 | 49.19 | 1.69 | 2.74 | 0.85 |
| 1 | 4 | 9 | 2 | 1 | 1.2 | 76.86 | 1.06 | 2.03 | 0.6 |
| 1 | 2 | 9 | 2 | 1 | 1.2 | 11.82 | 1.64 | 1.57 | 0.51 |
| 1 | 4 | 15 | 2 | 1 | 1.6 | 68.38 | 1.52 | 2.84 | 0.83 |
| 1 | 2 | 9 | 1.1 | 0 | 1.6 | 0.38 | 0.7 | 0 | 0 |
| 1 | 4 | 9 | 1.1 | 1 | 1.2 | 10.34 | 0.5 | 1.18 | 0.5 |
| 1 | 4 | 9 | 2 | 1 | 1.6 | 155.98 | 0.98 | 1.81 | 0.48 |
| 1 | 2 | 15 | 1.1 | 1 | 1.2 | 1.88 | 0.67 | 1.24 | 0.39 |
| 1 | 2 | 15 | 1.1 | 0 | 1.2 | 1.69 | 1.4 | 1.16 | 0.38 |
| 1 | 2 | 9 | 2 | 0 | 1.6 | 26.69 | 0.83 | 1.64 | 0.44 |
| 1 | 4 | 15 | 1.1 | 0 | 1.2 | 5.48 | 0.78 | 1.77 | 0.68 |
| 1 | 2 | 15 | 2 | 0 | 1.6 | 15.21 | 1.12 | 2.46 | 0.77 |
| 0 | 3 | 12 | 1.55 | 0.5 | 1.4 | 19.24 | 0 | 2.02 | 0.66 |
| 1 | 4 | 9 | 1.1 | 1 | 1.6 | 15.98 | 1.89 | 1.15 | 0.42 |
| 1 | 4 | 9 | 1.1 | 0 | 1.2 | 8.95 | 1.43 | 1.14 | 0.44 |
| 1 | 2 | 15 | 1.1 | 1 | 1.6 | 4.11 | 1.87 | 1.57 | 0.55 |
| 1 | 4 | 15 | 1.1 | 1 | 1.6 | 12.83 | 2.01 | 1.82 | 0.64 |
| 1 | 2 | 15 | 1.1 | 0 | 1.6 | 2.69 | 0 | 1.31 | 0.4 |
| 1 | 4 | 9 | 2 | 0 | 1.6 | 142.06 | 0.68 | 1.99 | 0.48 |
| 1 | 4 | 9 | 1.1 | 0 | 1.6 | 94.11 | 0.56 | 1.99 | 0.59 |
| 1 | 2 | 9 | 1.1 | 1 | 1.2 | 2.22 | 1.28 | 1.19 | 0.19 |
| 1 | 2 | 15 | 2 | 1 | 1.2 | 7.37 | 1.0 | 2.1 | 0.82 |
| 1 | 4 | 15 | 1.1 | 1 | 1.2 | 0.58 | 1.33 | 0.19 | 0.19 |
| 1 | 2 | 9 | 2 | 0 | 1.2 | 13.82 | 1.16 | 1.62 | 0.5 |
| 1 | 2 | 9 | 2 | 1 | 1.6 | 18.53 | 1.31 | 1.39 | 0.41 |
| 1 | 4 | 9 | 2 | 0 | 1.2 | 4.32 | 1.09 | 1.8 | 0.78 |
| 1 | 2 | 15 | 2 | 0 | 1.2 | 6.94 | 1.47 | 2.48 | 0.84 |
| 1 | 2 | 9 | 1.1 | 0 | 1.2 | 2.65 | 1.18 | 0.71 | 0.19 |
| 1 | 4 | 15 | 2 | 0 | 1.2 | 21.11 | 0.52 | 3.06 | 1.2 |
| 1 | 4 | 15 | 1.1 | 0 | 1.6 | 17.43 | 1.0 | 1.82 | 0.6 |
| 1 | 2 | 9 | 1.1 | 1 | 1.6 | 7.20 | 1.86 | 0.92 | 0.33 |
| 1 | 2 | 15 | 2 | 1 | 1.6 | 10.51 | 0.91 | 2.19 | 0.67 |
| 1 | 4 | 15 | 2 | 1 | 1.2 | 27.24 | 1.17 | 3.09 | 1.18 |

### Example 5

### Preparation of Naltrexone—Kl as Catalyst and NMP/Water as Solvent

A mixture of 10.02 g noroxymorphone, 4.39 g of NaHCO₃, 14.5 g of Kl, 4.9 g of chloromethylcyclopropane, 1-methyl-2-pyrrolidinone (36 mL) and water (4 mL) was heated at 85-90° C. for 2 h. While cooling, 120 mL of water was added with mechanical stirring. To dissolve the precipitate, the mixture was stirred with 50 mL chloroform. The layers were separated. The water layer was extracted with 20 mL chloroform. The combined chloroform mixture was extracted with 40 mL of 0.5 M hydrochloric acid, and then with 20 mL of 1.0 M hydrochloric acid. The acid extracts were combined and diluted to 100 mL. Concentrated ammonium hydroxide was added to give a pH over 9. Cooling and filtration, followed by washing with water gave a solid. Drying in vacuo gave 9.65 g of naltrexone monohydrate. The assay was 91.7% naltrexone base.

### Example 6

### Preparation of Naltrexone—Nabr as Catalyst and NMP/Water as Solvent

A mixture of 10.00 g noroxymorphone, 4.5 g of NaHCO₃, 9.1 g of NaBr, 2.5 g of chloromethylcyclopropane, 1-methyl-2-pyrrolidinone (36 mL), and water (4 mL) was heated at 85° C. for 2.5 h. To the warm mixture was added over 5 minutes 120 mL of water. Cooling in an ice-bath followed by filtering gave the product. Drying in vacuo gave 9.27 g of naltrexone base monohydrate. It assayed at 90.9% naltrexone.

### Example 7

### Preparation of Naltrexone—NaBr as Catalyst and DMF/Water as Solvent

A mixture of 5.04 g noroxymorphone, 2.2 g of NaHCO₃, 5.6 g of NaBr, 2.5 g of chloromethylcyclopropane, dimethylformamide (19 mL), and water (1 mL) was heated at 85° C. for 4 h. To the warm mixture was added over 5 minutes 60 mL of water. Cooling in an ice-bath followed by filtering gave the product. Drying in vacuo gave 4.21 g of naltrexone base monohydrate. It assayed at 94.6% naltrexone.

### Example 8

### Preparation of Naltrexone—Nabr as Catalyst and DMF/Water as Solvent

A mixture of 5.03 g noroxymorphone, 2.2 g of NaHCO₃, 5.6 g of NaBr, 2.5 g of chloromethylcyclopropane, and dimethylformamide (19 mL), and water (1 mL) was heated at 85° C. for 4 h. To the warm mixture was added over 5 minutes 60 mL of water. To the mixture was added 2 mL of concentrated ammonium hydroxide to give a pH over 10. Cooling and filtration gave 4.89 g of naltrexone monohydrate (assay 89.4%). The filtrate was extracted with 50 mL of chloroform. Extraction with 16 mL of 0.5 M HCl, followed by basification with ammonium hydroxide gave 0.66 g of additional naltrexone monohydrate. Adjusting for assay, a total of 5.0 g of naltrexone was obtained (83%).

### Example 9

### Preparation of Naltrexone—NaBr as Catalyst and DMAC/Water as Solvent

A mixture of 5.08 g noroxymorphone, 2.2 g of NaHCO₃, 5.6 g of NaBr, 2.5 g of chloromethylcyclopropane, dimethylacetamide (DMAC) (19 mL), and water (1 mL) was heated at 85° C. for 3 h. To the warm mixture was added over 5 minutes 60 mL of water. The pH was adjusted to above 9 with 2 mL of concentrated ammonium hydroxide. Cooling in an ice-bath followed by filtering and washing (10 mL water) gave the product. Drying in vacuo gave 5.77 g of naltrexone base monohydrate. It assayed at 91.5% naltrexone.

### Example 10

### Preparation of Naltrexone Nabr as Catalyst and DMAC/Water as Solvent

A mixture of 10.01 g noroxymorphone, 4.4 g of NaHCO₃, 11.2 g of NaBr, 5.0 g of chloromethylcyclopropane, dimethylacetamide (38 mL), and water (2 mL) was heated at 85° C. for 2.5 h. To the warm mixture was added over 5 minutes 120 mL of water. The pH was adjusted to above 10 with 4 mL of concentrated ammonium hydroxide. Cooling in an ice-bath followed by filtering and washing (20 mL water) gave the product. Drying in vacuo gave 11.06 g of naltrexone base monohydrate. It assayed at 94.1% naltrexone. Chloroform extraction followed by addition of hydrochloric acid essentially as described in Example 4 gave 0.26 g of additional naltrexone monohydrate.

### Example 11

### Preparation of Naltrexone—NaBr as Catalyst, Mixed Bases, and DMAC/Water as Solvent

A mixture of 5.02 g noroxymorphone, 1.11 g Na₂CO₃, 2.04 g of NaHCO₃, 5.6 g of NaBr, 2.5 g of chloromethylcyclopropane, dimethylacetamide (19 mL), and water (1 mL) was heated at 85° C. for 2.5 h. To the warm mixture was added over 5 minutes 60 mL of water. The pH was adjusted to above 9 with 1 mL of concentrated ammonium hydroxide. Cooling in an ice-bath followed by filtering and washing (2×10 mL water) gave the product. Drying in vacuo gave 5.63 g of naltrexone base monohydrate. It assayed at 92.3% naltrexone.

### Example 12

### Preparation of Naltrexone—LiBr as Catalyst and DMAC/Water as Solvent

A mixture of 5.03 g noroxymorphone, 2.25 g of NaHCO₃, 5.72 g of LiBr—H₂O, 2.5 g of chloromethylcyclopropane, dimethylacetamide (19 mL), and water (1 mL) was heated at 85° C. for 3 h. HPLC indicated significant unreacted noroxymorphone left. To the mixture was added 1.0 g chloromethylcyclopropane and heated at 85° C. for 1 h further. To the warm mixture was added over 5 minutes 60 mL of water. The pH was adjusted to above 9 with 1 mL of concentrated ammonium hydroxide. Cooling in an ice-bath followed by filtering and washing (2×10 mL water) gave the product, 4.82 g. Assay indicated 61.7% naltrexone.

### Example 13

### Preparation of Naltrexone NaBr/Bu₄NBr as Catalyst and DMAC/Water as Solvent

A mixture of 5.03 g noroxymorphone, 2.25 g of NaHCO₃, 3.6 g NaBr, 5.64 g of Bu₄NBr, 2.6 g of chloromethylcyclopropane, dimethylacetamide (18 mL), and water (2 mL) was heated at 85° C. for 2.5 h. To the warm mixture was added over 5 minutes 60 mL of water. The pH was adjusted to above 9 with 2 mL of concentrated ammonium hydroxide. Cooling in an ice-bath followed by filtering and washing (2×10 mL water) gave the product. Drying in vacuo gave 5.57 g of naltrexone base monohydrate. It assayed at 86.7% naltrexone.

### Example 14

### Preparation of Naltrexone—NaBr/Bu₄NBr as Catalyst and DMAC/Water as Solvent

A mixture of 5.01 g noroxymorphone, 2.23 g of NaHCO₃, 4.53 g NaBr, 2.83 g of Bu₄NBr, 2.55 g of chloromethylcyclopropane, dimethylacetamide (18 mL), and water (2 mL) was heated at 85° C. for 3.5 h. To the warm mixture was added over 5 minutes 60 mL of water. The pH was adjusted to above 9 with 2 mL of concentrated ammonium hydroxide. Cooling in an ice-bath followed by filtering and washing (2×10 mL water) gave the product. Drying in vacuo gave 5.45 g of naltrexone base monohydrate. It assayed at 92.8% naltrexone.

### Example 15

### Preparation of Naltrexone—NaBr/Me₄NBr as Catalyst and DMAC/Water as Solvent

A mixture of 5.00 g noroxymorphone, 2.28 g of NaHCO₃, 3.63 g NaBr, 2.68 g of Me₄NBr, 2.55 g of chloromethylcyclopropane, dimethylacetamide (18 mL), and water (2 mL) was heated at 85° C. for 2 h. To the warm mixture was added over 5 minutes 60 mL of water. The pH was adjusted to above 9 with 2 mL of concentrated ammonium hydroxide. Cooling in an ice-bath followed by filtering and washing (2×10 mL water) gave the product. Drying in vacuo gave 5.65 g of naltrexone base monohydrate. It assayed at 91.3% naltrexone.

### Example 16

### Preparation of Naltrexone—Me₄NBr as Catalyst and DMAC/Water as Solvent

A mixture of 5.00 g noroxymorphone, 2.30 g of NaHCO₃, 8.10 g of Me₄NBr, 2.5 g of chloromethylcyclopropane, dimethylacetamide (18 mL), and water (2 mL) was heated at 85° C. for 5 h. To the warm mixture was added over 5 minutes 60 mL of water. The pH was adjusted to above 9 with 2 mL of concentrated ammonium hydroxide. Cooling in an ice-bath followed by filtering and washing (2×10 mL water) gave the product. Drying in vacuo gave 4.26 g of product which was mainly naltrexone base monohydrate. It contained 70% naltrexone and 27% noroxymorphone by HPLC.

### Example 17

### Preparation of Naltrexone—NaBr/Bu₄NBr as Catalyst and NMP/Water as Solvent

A mixture of 5.03 g noroxymorphone, 2.28 g of NaHCO₃, 3.65 g NaBr, 5.68 g of Bu₄NBr, 2.5 g of chloromethylcyclopropane, 1-methyl-2-pyrrolidinone (18 mL), and water (2 mL) was heated at 85° C. for 2.5 h. To the warm mixture was added over 5 minutes 60 mL of water. The pH was adjusted to above 9 with 2 mL of concentrated ammonium hydroxide. Cooling in an ice-bath followed by filtering and washing (2×20 mL water) gave the off-colored solid as a crude product. It was then redissolved in 50 mL of water with 4 mL of 1.0 M HCl. Precipitation with ammonium hydroxide gave the improved solid. Drying in vacuo gave 5.23 g of naltrexone base monohydrate. It assayed at 91.7% naltrexone.

### Example 18

### Preparation of Naltrexone NaBr/Bu₃NMeBr as Catalyst and DMAC/Water as Solvent

A mixture of 5.02 g noroxymorphone, 2.31 g of NaHCO₃, 3.67 g NaBr, 4.93 g of Bu₃NMeBr, 2.5 g of chloromethylcyclopropane, dimethylacetamide (18 mL), and water (2 mL) was heated at 85° C. for 2.5 h. To the warm mixture was added over 5 minutes 60 mL of water. The pH was adjusted to above 9 with 2 mL of concentrated ammonium hydroxide. Cooling in an ice-bath followed by filtering and washing (2×10 mL water) gave the solid. Drying in vacuo gave 5.70 g of naltrexone base monohydrate. It assayed at 96.0% naltrexone.

### Example 19

### Preparation of Naloxone

The following reaction scheme depicts the synthesis of naloxone:

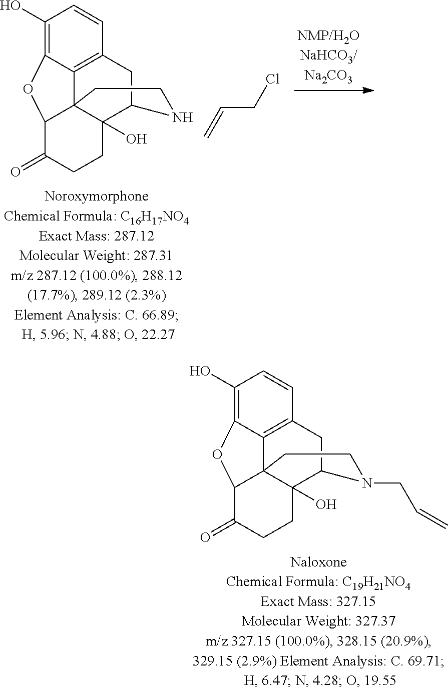

1.0 g (3.48 mmol, 1 eq) of noroxymorphone was added to a 25 mL round bottom flask, equipped with magnetic stirring. Na₂CO₃ (0.7 eq, 0.26 g) and NaHCO₃ (1.3 eq, 0.38 g) were added into the flask, followed by NaBr salt (2.8 eq, 1.0 g). The mixed solvent (1/9 H₂O/NMP, 4.0 mL) and allyl chloride (1.38 eq, 0.396 mL) were added successively into the mixture, which became a white slurry. The flask was then sealed with a pressure cap, and stirred in a 30° C. oil bath for 16 hours. The reaction mixture was then quenched with water. Chromatography showed over 97% conversion of noroxymorphone to naloxone with <2% of related impurity. After work up, 1.1 g of dry off-white solid was recovered.

### Example 20

### Preparation of Buprenorphine

Buprenorphine was synthesized according to the following reaction scheme:

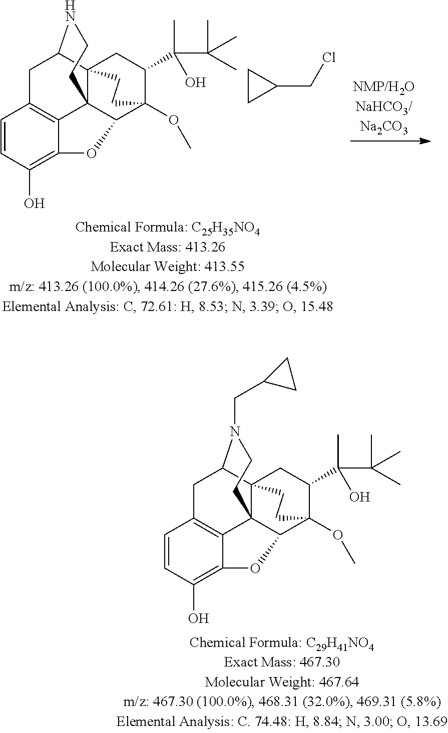

1.0 g (2.42 mmol, 1 eq) of norbuprenorphine was added to a 25 mL three neck flask, equipped with magnetic stirring. Na₂CO₃ (0.7 eq, 0.26 g) and NaHCO₃ (1.3 eq, 0.38 g) were added into the flask, followed by NaBr salt (2.8 eq, 0.7 g). The mixed solvent (1/9 H₂O/NMP, 4.0 mL) and cyclopropylmethyl chloride (1.38 eq, 0.316 mL) were added successively into the mixture, which became a dark brown slurry. The mixture was heated to 70° C. for 5 hours before the reaction was quenched with water. Chromatography showed 85% conversion of norbuprenorphine to the buprenorphine. After work up, 0.85 g of dry tan solid was recovered.

What is claimed is:

1. A process for preparing a compound comprising Formula (II): 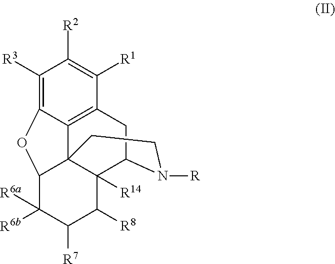
the process comprising:
contacting a compound comprising Formula (I) with R-chloride and a bromide or iodide salt to form the compound comprising Formula (II), the compound of Formula (I) comprising: 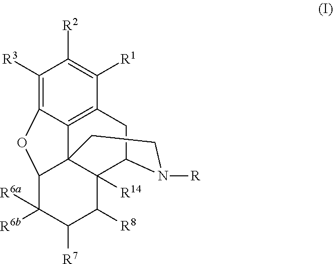
wherein:
R is chosen from hydrocarbyl and substituted hydrocarbyl;
R¹ and R² are independently chosen from hydrogen, halogen, hydrocarbyl, and substituted hydrocarbyl;
R³, R⁷, and R⁸ are independently chosen from hydrogen, {—}OR¹¹, hydrocarbyl, and substituted hydrocarbyl;
R^{6a} and R^{6b} are independently chosen from hydrogen, {—}OR¹¹, hydrocarbyl, substituted hydrocarbyl, combining with R¹⁴ to from an alkeno bridge, and together forming {═}O or {═}CH₂;
R¹¹ is chosen from hydrogen, hydrocarbyl, substituted hydrocarbyl, and a hydroxy protecting group; and
R¹⁴ is chosen from hydrogen, {—}OR¹¹; hydrocarbyl, substituted hydrocarbyl, and combining with R^{6a} or R^{6b} to form an alkeno bridge.

2. The process of claim 1, wherein R is chosen from alkyl, alkenyl, alkynyl, aryl, substituted alkyl, substituted alkenyl, substituted alkynyl, and substituted aryl.

3. The process of claim 1, wherein R¹, R², and R⁸ are hydrogen; and R³ is chosen from alkoxy, hydroxy, and protected hydroxyl.

4. The process of claim 3, wherein R^{6a} and R^{6b} together form {═O}; R⁷ is hydrogen; and R¹⁴ is hydroxy.

5. The process of claim 3, wherein one of R^{6a} or R^{6b} combines with R¹⁴ to form an ethano bridge and the other is methoxy, and R⁷ is {—}CH(CH₃)(OH)CH(CH₃)₃.

6. The process of claim 1, wherein the compound comprising Formula (I) is noroxymorphone.

7. The process of claim 1, wherein the compound comprising Formula (I) is norbuprenorphine.

8. The process of claim 1, wherein R is chosen from methyl, cyclopropylmethyl, cyclobutylmethyl, and allyl.

9. The process of claim 1, wherein the bromide or iodide salt is chosen from NaBr, KBr, LiBr, R¹R²R³R⁴NBr, NaI, KI, Lil, R¹R²R³R⁴NI, and combinations thereof, wherein R¹, R², R³, and R⁴ are independently alkyl.

10. The process of claim 1, wherein the molar ratio of the compound comprising Formula (I) to R-chloride to the bromide or iodide salt is from about 1:0.8:1.1 to about 1:2:5.

11. The process of claim 1, wherein the reaction is conducted in the presence of a proton acceptor having a pKa from about 7 to about 13.

12. The process of claim 11, wherein the proton acceptor is chosen from a bicarbonate, a carbonate, a phosphate, and an organic base.

13. The process of claim 11, wherein the molar ratio of the compound comprising Formula (I) to the proton acceptor is from about 1:1 to about 1:4.

14. The process of claim 1, wherein the reaction is conducted at a pH from about 8 to about 10.

15. The process of claim 1, wherein the reaction is conducted in the presence of a solvent chosen from N,N-dimethylformamide (DMF), N,N-dimethylacetamide (DMAC), and N-methyl-2-pyrrolidinone (NMP).

16. The process of claim 15, wherein the solvent further comprise from about 1% to about 15% by weight of water.

17. The process of claim 15, wherein the weight ratio of the solvent to the compound comprising Formula (I) is from about 2:1 to about 10:1.

18. The process of claim 1, wherein the reaction is conducted at a temperature from about 20° C. to about 120° C.

19. The process of claim 1, wherein the yield of the compound comprising Formula (II) is at least about 60% by weight.

20. The process of claim 1, wherein the optical activity of the compounds comprising Formulas (I) or (II) is (−) or (+), and the configuration of C-5, C-13, C-14, and C-9, respectively, is chosen from RRRR, RRRS, RRSR, RSRR, SRRR, RRSS, RSSR, SSRR, SRRS, SRSR, RSRS, RSSS, SRSS, SSRS, SSSR, and SSSS, provided that C-15 and C-16 are both either on the alpha face or the beta face of the molecule.
